(19) United States
(12) Patent Application Publication
(10) Pub. No.: US 2021/0284603 A1
(43) Pub. Date: Sep. 16, 2021

(54) METHOD FOR EXTRACTING DIISOCYANATES FROM DISTILLATION RESIDUES

(71) Applicant: Covestro Deutschland AG, Leverkusen (DE)

(72) Inventors: Claudine STOYE, Köln (DE); Thomas GUTTMANN, Langenfeld (DE); Sascha TADJBACH, Bergisch Gladbach (DE); Thomas VOIGT, Langenfeld (DE); Irene Christina LATORRE MARTINEZ, Leverkusen (DE); Joerg MORAWSKI, Leverkusen (DE)

(21) Appl. No.: 16/478,393

(22) PCT Filed: Jan. 17, 2018
(86) PCT No.: PCT/EP2018/051087

§ 371 Date: Jul. 16, 2019

(30) Foreign Application Priority Data
Jan. 18, 2017 (EP) 17151971.3

(51) Int. Cl. C07C 263/20; C07C 265/14; C08G 18/76; C08G 18/42; B01D 1/24; B01D 3/10

(52) U.S. Cl. CPC C07C 263/20; C07C 265/14; B01D 3/10; C08G 18/42; B01D 1/24; C08G 18/7678

(57) ABSTRACT

The invention relates to a method for extracting diisocyanates that are solid at room temperature from a distillation residue, comprising the following steps: (i) providing at least one residue containing diisocyanates that are solid at room temperature, and (ii) treating the residue at a temperature of ≥120° C. to <200° C. so as to obtain diisocyanates that are solid at room temperature, the residue provided in step (i) containing ≥32 to ≤60 wt. % of bitumen and the treatment in step (ii) being carried out in at least one thin-film evaporator and/or falling-film evaporator.

The present invention relates to a process for recovering room temperature solid diisocyanates from a distillation residue. The invention further provides the room temperature solid diisocyanate obtainable by this process. In addition, the invention further provides for the use of a thin-film evaporator, a composition comprising the room temperature solid diisocyanate, and a process for producing an elastomer and the elastomer itself.

The industrial scale preparation of diisocyanates by reacting amines with phosgene in solvents is known and described in detail in the literature (Ullmanns Encyklopädie der technischen Chemie [Ullmann's Encyclopedia of Industrial Chemistry], 4th edition, volume 13, pages 347-357, Verlag Chemie, GmbH, D-6940 Weinheim, 1977 or else EP 1 575 908 A1). Usually, the production of pure distilled diisocyanates in the distillation processes affords a by-product stream that has to be disposed of as residue after widely possible distillative removal of free isocyanates. Depending on the chemical nature of the diisocyanate prepared, this residue contains a considerable proportion of monomeric diisocyanate. It was thus desirable to be able to recover the monomeric diisocyanate to the best possible degree from the residue in order to be able to increase the overall yield without worsening the overall economic viability of the process.

If the diisocyanate prepared is liquid at room temperature, the handling of the residue is simplified as a result. By contrast, the recovery of room temperature solid diisocyanates is particularly difficult since the residue has a much higher viscosity extending as far as a solid state and can thus be treated only in a technically complex manner. A simple increase in temperature has not been successful since, although it is thus possible to lower the viscosity, there are unwanted side reactions—for example oligomerizations—of the diisocyanate to be isolated.

A room temperature solid diisocyanate that has found broad industrial use is naphthalene diisocyanate (NDI). As described at the outset, NDI can be prepared in principle by means of known processes, for example by phosgenation, from naphthalenediamine (NDA). The NDI prepared is isolated by known processes, leaving a residue containing a wide variety of different compounds from the phosgenation of NDA to NDI.

Since monomeric NDI is a solid that melts at 127° C. and already starts to sublime at 130° C., the workup and removal of the monomer constitutes a particular challenge compared to other isocyanates that are liquid at processing temperature, for example tolylene diisocyanate (TDI).

WO 2007/036479 A1 discloses a general process for purifying isocyanate-containing residues, in which it is less preferably possible also to use naphthyl diisocyanate. No working examples are disclosed. The distillation takes place at temperatures between 210° C. and 330° C. At such high temperatures, there can be increased occurrence of oligomerization reactions. Moreover, energy expenditure is disproportionately high.

EP 0 626 368 A1 describes a process for preparing pure distilled isocyanates in which the residue is admixed with 2% to 50% by weight of high-boiling hydrocarbons and the isocyanate is extracted from this residue at temperatures of 160° C. to 280° C. Kneader-driers are used in this process, which mean relatively complex technology with mechanically moving parts. Since the continuous discharge of free-flowing material has to be assured in the case of this technology, the nature of the residue is crucial and greatly restricts utilization.

U.S. Pat. No. 3,694,323 discloses a process for recovering an isocyanate from its phosgenation residue with the aid of what is called an isocyanate exchange medium which has a higher boiling point than the isocyanate to be purified and hence lowers the viscosity of the phosgenation residue and enables purification. However, a disadvantage of this process is that the purified isocyanate is contaminated by the isocyanate exchange medium and the process has a comparatively high energy requirement since temperatures between 190° C. and 250° C. are required in the examples for the purification of TDI.

It was therefore an object of the present invention to provide a process for obtaining room temperature solid diisocyanates, especially naphthalene diisocyanate, from a distillation residue, which has a lower energy consumption than in the prior art and in which an elevated yield of recovered room temperature solid diisocyanates can simultaneously be achieved.

This object was achieved in accordance with the invention by a process for recovering room temperature solid diisocyanates from a distillation residue, comprising the following steps:
(i) providing at least one residue containing room temperature solid diisocyanates and
(ii) treating the residue at a temperature of ≥120° C. to <200° C. to obtain room temperature solid diisocyanates,
   wherein the residue provided in step (i) contains ≥32% to ≤60% by weight of bitumen and the treatment in step (ii) is effected in at least one thin-film evaporator and/or falling-film evaporator.

In the present context, “room temperature solid diisocyanates” is understood to mean that the diisocyanates are in the solid state of matter at 23° C. and standard pressure.

In the present context, naphthalene diisocyanate is understood as an umbrella term for the possible isomers or mixtures thereof. Examples of such isomers are naphthalene 1,5-diisocyanate or naphthalene 1,8-diisocyanate.

According to the invention the terms “comprising” or “containing” preferably mean “consisting essentially of” and particularly preferably mean “consisting of”.

Suitable thin-film evaporators are, for example, star blade rotor thin-film evaporators or wiper-blade thin-film evaporators or short-path evaporators.

Suitable falling-film evaporators are, for example, shell and tube downpipe evaporators or helical tube evaporators.

In a first preferred embodiment, the temperature in step (ii) is ≥130° C. to <190° C., preferably ≥140° C. to ≤180° C. and more preferably ≥150° C. to ≤165° C. This gives rise to the advantage that the formation of by-products, i.e. oligomerization during the distillation, can be very substantially suppressed.

In a further preferred embodiment, the residue provided in step (i) contains ≥35% to 555% by weight, preferably ≥40% to 50% by weight, of bitumen. This gives rise to the additional advantage that the yield of room temperature solid diisocyanate recovered can be further increased and the mixture is kept in liquid form throughout the distillation process.

Bitumen is known to the person skilled in the art and can be sourced as 70/100 bitumen from Shell, for example. In the present context, preference is given to using 70/100 bitumen from Shell.

Particular preference is given to combinations of the two aforementioned preferred embodiments since they can bring an additional increase in yield.

In a further preferred embodiment, the room temperature solid diisocyanate is naphthalene 1,5-diisocyanate, naphthalene 1,8-diisocyanate, phenylene 1,4-diisocyanate, tetralin diisocyanate, o-toluidine diisocyanate, durene diisocyanate, benzidine diisocyanate and/or anthrylene 1,4-diisocyanate, preferably naphthalene 1,5-diisocyanate, naphthalene 1,8-diisocyanate, phenylene 1,4-diisocyanate, tetralin diisocyanate and/or o-toluidine diisocyanate and more preferably naphthalene 1,5-diisocyanate and/or naphthalene 1,8-diisocyanate.

In principle, the room temperature solid diisocyanates can be prepared by any routes, for example by reaction of the corresponding diamines or salts thereof with phosgene. All that is important in each case is that the process utilized leaves at least one residue containing room temperature solid diisocyanates that can then be used in step (i) of the invention.

More preferably, the residue containing room temperature solid diisocyanates which is provided in step (i) comes from the phosgenation of the corresponding diamines, preferably from the liquid phase phosgenation of the corresponding diamines.

In the present context, the term “corresponding diamine” is in each case understood to mean the room temperature solid diisocyanate that is to be prepared in which the two isocyanate groups have been exchanged for amino groups. By way of example, naphthalene 1,5-diamine is the corresponding diamine of naphthalene 1,5-diisocyanate. When the room temperature solid diisocyanate is an isomer mixture, a corresponding isomer mixture of diamines is used.

The continuous preparation of organic isocyanates by reaction of primary organic amines with phosgene has been described many times and is performed on the industrial scale (see, for example, Ullmanns Encyclopedia of Industrial Chemistry, Wiley-VCH Verlag GmbH & Co KGaA, Weinheim, Online ISBN: 9783527306732, DOI: 10.1002/14356007.a14_611, p. 63 If (2012)). Particular preference is given to preparing the room temperature solid diisocyanate from the corresponding diamine using phosgene by the process known from WO 2014/044699 A1, which comprises the following steps:

(A) preparing a suspension of the corresponding diamine in an inert solvent, where the diamine is distributed in the solvent by means of a dynamic mixing unit,
(B) phosgenating the diamine suspended in the inert solvent to obtain the respective diisocyanate,
wherein the dynamic mixing unit in step (A) is selected from the group consisting of dispersing disks and rotor-stator systems, preferably rotor-stator systems, more preferably colloid mills, toothed dispersing machines and three-roll mills. Very particular preference is given to toothed dispersing machines as dynamic mixing units.

The dispersing disks and rotor-stator systems mentioned in the above paragraph have the same meaning as at page 4 line 17 to page 5 line 11 of WO 2014/044699 A1.

Suitable inert solvents are aromatic solvents, which may also be halogenated. Examples of these are toluene, monochlorobenzene, o-, m- or p-dichlorobenzene, trichlorobenzene, chlorotoluenes, chloroxylenes, chloroethylbenzene, chloronaphthalenes, chlorodiphenyls, xylenes, decahydronaphthalene, benzene or mixtures of the above solvents. Further examples of suitable organic solvents are methylene chloride, perchloroethylene, hexane, diethyl isophthalate, tetrahydrofuran (THF), dioxane, trichlorofluoromethane, butyl acetate and dimethylformamide (DMF). Preference is given to using monochlorobenzene or o-dichlorobenzene or a mixture of the two; particular preference is given to using monochlorobenzene.

Phosgene is used in excess in the reaction in step (B). This means that more than one mole of phosgene is used per mole of amine groups. The molar ratio of phosgene to amine groups is accordingly from 1.01:1 to 20:1, preferably 1.1:1 to 10:1, more preferably 1.1:1 to 5.0:1. If necessary, further phosgene or phosgene solution can be supplied to the reaction mixture during the reaction in order to maintain a sufficient excess of phosgene or to compensate for loss of phosgene.

The reaction can be performed continuously and batchwise. Useful reactors include stirred tanks, tubular reactors, spray towers or else loop reactors. In principle, it is alternatively possible to utilize other designs that are not listed here by way of example. Preference is given to batchwise operation.

The reaction can be conducted until complete conversion to the isocyanate within the first reaction stage. Alternatively, it may be advantageous or necessary to conduct a partial conversion, especially of residues of amine hydrochloride, in a downstream reactor. The downstream reactor may comprise customary reactor designs with different degrees of backmixing, such as stirred tanks, loop reactors or tubular reactors. It may also be advantageous to divide the reaction mixture into substreams according to its particle size distribution and feed them separately to one or more downstream reactors. Useful designs for the removal include known apparatuses such as filters, cyclones or gravitational separators, for example. The substreams may be treated before or during the reaction by appropriate mechanical methods of adjusting the particle size, for example by grinding.

The unconverted phosgene is usually recycled, optionally after purification, and reused for phosgenation.

Methods available for separating the diisocyanate from the solvent include those known to the person skilled in the art, for example crystallization, sublimation or distillation, optionally with addition of seed crystals or azeotroping agents, for example. Preference is given to using a process comprising crystallization or distillation.

However, the high-viscosity or even solid residue that remains after the purification still contains a variably high proportion of diisocyanates. This residue is an example of a residue containing room temperature solid isocyanates that is to be used in step (i) of the invention.

At least an appropriate amount of bitumen is then added to this residue or else to corresponding residues obtained by other methods than the method disclosed as being particularly preferred above, and the residue thus provided is treated in step (ii) of the process of the invention.

In a further preferred embodiment, the residue in the treatment in step (ii) contains ≥0% by weight to ≤4% by weight, preferably ≥0.001% by weight to ≤2% by weight and more preferably ≥0.01% by weight to 51% by weight, based in each case on the total amount of the residue containing room temperature solid diisocyanates, of monomeric diisocyanates having a boiling temperature above the boiling temperature of the room temperature solid diisocyanate. This is particularly advantageous since, even without this addition, a high yield can be achieved in the recovery and the purified room temperature solid diisocyanate thus remains very substantially free of these impurities.

The percentages by weight of monomeric diisocyanates having a boiling temperature above the boiling temperature of the room temperature solid diisocyanate are determined by gas chromatography by means of an FID detector, preferably using an Optima 5 column and the following parameters: split rate: 8.31:1 mL/min; flow rate: 96.4 mL/min; pressure: 0.7 bar, carrier gas: helium, injection volume: 1 μL, inliner: straight split liner filled with Carbofritt, equating the area percentage with the percentage by weight in the evaluation.

In a further preferred embodiment, the residue has an average residence time in the treatment in step (ii) of ≥1 to ≤15 minutes, preferably of ≥1 to ≤10 minutes and more preferably of ≥1 to ≤5 minutes in the at least one thin-film evaporator and/or falling-film evaporator. This results in the advantage that the likelihood of unwanted side reactions—for example oligomerizations—can be reduced further.

In a further preferred embodiment, the treatment in step (ii) takes place at a pressure of ≥0.4 mbar to ≤4.0 mbar, preferably of ≥0.7 mbar to ≤2 mbar and more preferably of ≥0.8 mbar to ≤1.5 mbar.

In a further preferred embodiment, the treatment in step (ii) takes place at a coolant temperature below the melting point of the room temperature solid diisocyanate.

The abovementioned preferred embodiments can also be combined with one another, which can lead to a further reduction in the tendency to form by-products.

Preferably, the treatment in step (ii) recovers ≥60% by weight, preferably ≥70% by weight and most preferably ≥80% by weight of the room temperature solid diisocyanates that are still present in the residue in step (i).

The present invention further provides the room temperature solid diisocyanates obtained or obtainable by the process of the invention, and they can be used, for example, for production of high-performance elastomers, for example Vulkollan®.

None of the known processes for treatment of residues containing room temperature solid diisocyanates to date have used thin-film evaporators and/or falling-film evaporators. Thus, in the present context, the use of a thin-film evaporator and/or falling-film evaporator for recovery of room temperature solid diisocyanates from a residue containing room temperature solid diisocyanates is disclosed. Particular preference is given to the use of a thin-film evaporator and/or falling-film evaporator in the treatment of a distillation residue containing room temperature solid diisocyanates for increasing the yield of recovered diisocyanate and/or for reducing by-product formation.

The present invention therefore further provides for the use of a thin-film evaporator for recovering aromatic diisocyanates that are solid at room temperature from a residue containing aromatic diisocyanates that are solid at room temperature.

“Aromatic” diisocyanates are understood here to mean diisocyanates in which both isocyanate groups are bonded to aromatic carbon atoms. Preferably, the room temperature solid diisocyanates are selected from the group consisting of naphthalene 1,5-diisocyanate, naphthalene 1,8-diisocyanate, phenylene 1,4-diisocyanate, tetralin diisocyanate, o-toluidine diisocyanate, durene diisocyanate, benzidine diisocyanate and anthrylene 1,4-diisocyanate, preferably selected from the group consisting of naphthalene 1,5-diisocyanate, naphthalene 1,8-diisocyanate, phenylene 1,4-diisocyanate, tetralin diisocyanate and o-toluidine diisocyanate, and more preferably selected from the group consisting of naphthalene 1,5-diisocyanate and naphthalene 1,8-diisocyanate.

In the use of the invention, it is always particularly preferable that the residue is free of polyalkylene polyethers. The room temperature solid diisocyanates obtained by the process of the invention, even alone or else in mixtures with the portion obtained directly from the first purification stage after the phosgenation reaction, can be sent to all end uses familiar to the person skilled in the art. Particular mention should be made of further processing with NCO-reactive compounds such as polyols to give polyurethanes, optionally via prepolymers as intermediates.

These polyurethanes preferably have apparent densities of 200 kg/m³ to 1400 kg/m, more preferably of 600 kg/m³ to 1400 kg/m³ and most preferably of 800 kg/m³ to 1400 kg/m. Very particular preference is given to using cellular or bulk cast elastomers, more preferably polyester polyol-based cast elastomers.

The present invention thus further provides a composition comprising at least room temperature solid diisocyanate of the invention and at least one NCO-reactive compound, preferably at least one polyester polyol.

The composition may additionally comprise customary assistants and additives, for example rheology improvers (for example ethylene carbonate, propylene carbonate, dibasic esters, citric esters), stabilizers (for example Brønsted and Lewis acids, for instance hydrochloric acid, phosphoric acid, benzoyl chloride, organo mineral acids such as dibutyl phosphate, and also adipic acid, malic acid, succinic acid, pyruvic acid or citric acid), UV stabilizers (for example 2,6-dibutyl-4-methylphenol), hydrolysis stabilizers (for example sterically hindered carbodiimides), emulsifiers and catalysts (for example trialkylamines, diazabicyclooctane, tin dioctoate, dibutyltin dilaurate, N-alkylmorpholine, lead octoate, zinc octoate, tin octoate, calcium octoate, magnesium octoate, the corresponding naphthenates and p-nitrophenoxide and/or else mercury phenylneodecanoate) and fillers (for example chalk), dyes which may be incorporable into the polyurethane/polyurea to be formed at a later stage (which thus possess Zerewitinoff-active hydrogen atoms) and/or color pigments.

NCO-reactive compounds used may be any compounds known to those skilled in the art.

As NCO-reactive compounds may polyether polyols, polyester polyols, polycarbonate polyols and polyether amines having an average OH or NH functionality of at least 1.5, and short-chain polyols and polyamines (chain extenders or crosslinkers), as are sufficiently well known from the prior art. These may be, for example, low molecular weight diols (e.g. 1,2-ethanediol, 1,3- or 1,2-propanediol, 1,4-butanediol), triols (e.g. glycerol, trimethylolpropane) and tetraols (e.g. pentaerythritol), but also higher molecular weight polyhydroxyl compounds such as polyether polyols, polyester polyols, polycarbonate polyols, polysiloxane polyols, polyamines and polyether polyamines and polybutadiene polyols.

Polyether polyols are obtainable in a manner known per se, by alkoxylation of suitable starter molecules under base catalysis or using double metal cyanide compounds (DMC compounds). Suitable starter molecules for the preparation of polyether polyols are, for example, simple low molecular weight polyols, water, organic polyamines having at least two N—H bonds, or any desired mixtures of such starter molecules. Preferred starter molecules for preparation of polyether polyols by alkoxylation, especially by the DMC process, are especially simple polyols such as ethylene glycol, propylene 1,3-glycol and butane-1,4-diol, hexane-1,6-diol, neopentyl glycol, 2-ethylhexane-1,3-diol, glycerol, trimethylolpropane, pentaerythritol, and low molecular weight hydroxyl-containing esters of such polyols with dicarboxylic acids of the kind specified hereinafter by way of example, or low molecular weight ethoxylation or propoxylation products of such simple polyols, or any desired mixtures of such modified or unmodified alcohols. Alkylene oxides suitable for the alkoxylation are especially ethylene oxide and/or propylene oxide, which can be used in the alkoxylation in any sequence or else in a mixture.

Polyester polyols can prepare in a known manner by polycondensation of low molecular weight polycarboxylic acid derivatives, for example succinic acid, adipic acid, suberic acid, azelaic acid, sebacic acid, dodecanedioic acid, tetrahydrophthalic anhydride, hexahydrophthalic anhydride, tetrachlorophthalic anhydride, endomethylenetetrahydrophthalic anhydride, glutaric anhydride, maleic acid, maleic anhydride, fumaric acid, dimer fatty acid, trimer fatty acid, phthalic acid, phthalic anhydride, isophthalic acid, terephthalic acid, citric acid or trimellitic acid, with low molecular weight polyols, for example ethylene glycol, diethylene glycol, neopentyl glycol, hexanediol, butanediol, propylene glycol, glycerol, trimethylolpropane, 1,4-hydroxymethylcyclohexane, 2-methyl-1,3-propanediol, butane-1,2,4-triol, triethylene glycol, tetraethylene glycol, polyethylene glycol, dipropylene glycol, polypropylene glycol, dibutylene glycol and polybutylene glycol, or by ring-opening polymerization of cyclic carboxylic esters such as ε-caprolactone. In addition, it is also possible to polycondense hydroxycarboxylic acid derivatives, for example lactic acid, cinnamic acid or o-hydroxycaproic acid to give polyester polyols. However, it is also possible to use polyester polyols of oleochemical origin. Such polyester polyols can be prepared, for example, by full ring-opening of epoxidized triglycerides of an at least partly olefinically unsaturated fatty acid-containing fat mixture with one or more alcohols having 1 to 12 carbon atoms and subsequent partial transesterification of the triglyceride derivatives to alkyl ester polyols having 1 to 12 carbon atoms in the alkyl radical.

The NCO-reactive compound may contain short-chain polyols or polyamines as crosslinker component or chain extender. Typical chain extenders are diethyltoluenediamine (DETDA), 4,4′-methylenebis(2,6-diethyl)aniline (MDEA), 4,4′-methylenebis(2,6-diisopropyl)aniline (MDIPA), 4,4′-methylenebis(3-chloro-2,6-diethyl)aniline (MCDEA), dimethylthiotoluenediamine (DMTDA, Ethacure® 300), N,N′-di(sec-butyl)aminobiphenylmethane (DBMDA, Unilink® 4200) or N,N′-di-sec-butyl-p-phenylenediamine (Unilink® 4100), 3,3′-dichloro-4,4′-diaminodiphenylmethane (MBOCA), trimethylene glycol di-p-aminobenzoate (Polacure 740M). Aliphatic aminic chain extenders can likewise be used, or can be used in addition. Propane-1,3-diol, butane-1,4-diol, butane-2,3-diol, pentane-1,5-diol, hexane-1,6-diol and HQEE (hydroquinone di(β-hydroxyethyl) ether), and also water. Very particular preference is given to using butane-1,4-diol for bulk cast elastomers and water for cellular cast elastomers.

An overview of polyurethanes and their properties and uses is given, for example, in the Kunststofthandbuch [Plastics Handbook], volume 7, Polyurethane [Polyurethanes], 3rd newly revised edition, volume 193, edited by Prof. Dr. G. W. Becker and Prof. Dr. D. Braun (Carl-Hanser-Verlag, Munich, Vienna).

Preference is given to using NCO-terminated prepolymers having an NCO content of 2% to 15% by weight, very particularly of 2-10% by weight. The room temperature solid diisocyanate is preferably reacted with polyols of functionality 2 to 3, preferably 2, and OH number 28-112 mg KOH/g of substance to give prepolymers. Preference is given to using ester-based polyols. The NCO prepolymers thus prepared are either converted further directly or stored as storage-stable prepolymers in drums, for example, until they are ultimately used. Preference is given to using 1,5-NDI-based polyols. The production of the cast elastomers (molded articles) is advantageously conducted at an NCO/OH ratio of 0.7 to 1.30. In the case of cellular elastomers, the amount of the mixture introduced into the mold is typically such that the shaped bodies obtained have the density already described. The starting components are typically introduced into the mold at a temperature of 30 to 110° C. The degrees of densification are between 1.1 and 8, preferably between 2 and 6. The cellular elastomers are preferably produced by a low-pressure technique or especially the reactive injection molding technique (RIM) in open molds, preferably closed molds.

The reactive injection molding technique is described, for example, by H. Piechota and H. Röhr in “Integral Schaumstoffe” [Integral Foams], Carl Hanser-Verlag, Munich, Vienna 1975; D. J. Prepelka

and J. L. Wharton in Journal of Cellular Plastics, March/April 1975, pages 87 to 98 and U. Knipp in Journal of CellularPlastics, March/April 1973, pages 76-84.

Additives such as castor oil or carbodiimides (for example Stabaxols from Rheinchemie as hydrolysis stabilizer, 2,2′,6,6′-tetraisopropyldiphenylcarbodiimide is a known representative) can be added either to the polyol or to the prepolymer. Water, emulsifiers, catalysts and/or auxiliaries and/or additives commonly form the polyol component together with the polyol.

For better demolding, it is customary to provide the molds with external separating agents, for example wax- or silicone-based compounds or aqueous soap solutions. The demolded shaped bodies are typically subjected to subsequent heat treatment at temperatures of 70 to 120° C. for 1 to 48 hours.

Emulsifiers used are, for example, sulfonated fatty acids and further commonly known emulsifiers, for example polyglycol esters of fatty acids, alkylaryl polyglycol ethers, alkoxylates of fatty acids, preferably polyethylene glycol esters, polypropylene glycol esters, polyethylene-polypropylene glycol esters, ethoxylates and/or propoxylates of linoleic acid, linolenic acid, oleic acid, arachidonic acid, more preferably oleic acid ethoxylates. Alternatively, it is also possible to use polysiloxanes. Salts of fatty acids with amines, e.g. diethylammonium oleate, diethanolammonium stearate, diethanolammonium ricinoleate, salts of sulfonic acids, e.g. alkali metal or ammonium salts of dodecylbenzene- or dinaphthylmethanedisulfonic acid, are likewise preferred.

The sulfonated fatty acids can preferably be used as aqueous solutions, for example as a 50% solution. Typical known products are SV and SM additives from Rheinchemie, and WM additive from Rheinchemie as a nonaqueous emulsifier.

The process for producing the cellular PUR cast elastomers is conducted in the presence of water. The water acts both as crosslinker with formation of urea groups and as blowing agent on account of the reaction with isocyanate groups to form carbon dioxide. The amounts of water that can appropriately be used are 0.01% to 5% by weight, preferably 0.3% to 3.0% by weight, based on the weight of the polyol component. The water may be used entirely or partly in the form of the aqueous solutions of the sulfonated fatty acids.

The catalysts may be added individually or else in a blend with one another. These are preferably organometallic compounds such as tin(II) salts of organic carboxylic acids, e.g. tin(II) dioctoate, tin(II) dilaurate, dibutyltin diacetate and dibutyltin dilaurate, and tertiary amines such as tetramethylethylenediamine, N-methylmorpholine, diethylbenzylamine, triethylamine, dimethylcyclohexylamine, diazabicyclooctane, N,N′-dimethylpiperazine, N-methyl-N′-(4-N-dimethylamino)butylpiperazine, N,N,N′,N″,N″-pentamethyldiethylenetriamine or the like. Further useful catalysts include: amidines, for example 2,3-dimethyl-3,4,5,6-tetrahydropyrimidine, tris(dialkylaminoalkyl)-s-hexahydrotriazines, especially tris(N,N-dimethylaminopropyl)-s-hexahydrotriazine, tetraalkylammonium hydroxides, for example tetramethylammonium hydroxide, alkali metal hydroxides, for example sodium hydroxide, and alkali metal alkoxides, for example sodium methoxide and potassium isopropoxide, and alkali metal salts of long chain fatty acids having 10 to 20 carbon atoms and optionally pendant OH groups. According to the reactivity to be established, the catalysts are employed in amounts of 0.001% to 0.5% by weight, based on the isocyanate component.

The present invention further provides a process for producing an elastomer, in which at least one composition of the invention is cured, optionally while heating, and the elastomer produced or producible by this process.

Such cellular PUR cast elastomers, also referred to as shaped bodies, find use as damping elements in vehicle construction, for example in automobile construction, for example as overload springs, buffers, transverse link bearings, rear axle subframe bearings, stabilizer bearings, longitudinal strut bearings, suspension strut bearings, shock absorber bearings, or bearings for wishbones, and also as an emergency wheel on the rim, which has the effect that the vehicle, in the event of tire damage, runs on the cellular elastomer and remains controllable. The bulk cast elastomers can also be used as a coating for rolls, wheels and drums, squeegees, screens or hydrocyclones.

The present invention is more particularly elucidated hereinafter with reference to examples and comparative examples, but without restricting it thereto.

### EXAMPLES

All percentages are based on weight, unless stated otherwise.

The purity of the NDI was determined by gas chromatography. The measurements were effected using a Hewlett Packard HP 6890 with an FID detector and HP-Chemstation software using an Optima 5 column and the following parameters: split rate: 8.31:1 mL/min; flow rate: 96.4 mL/min; pressure: 0.7 bar, carrier gas: helium, injection volume: 1 μL, inliner straight split liner filled with Carbofritt.

The NDI residues before and after distillation were analyzed by means of GPC to DIN 55672-1:2007-08. The yield was then determined by subtracting the area percentage of the monomer still remaining from the area percentage of the original amount of monomer, which were determined in each case by GPC to DIN 55672-1:2007-08.

The bitumen used was sourced from Shell and corresponded to Shell 70/100 bitumen quality.

### Example 1 (Inventive)

A mixture containing 50% residue from the phosgenation of 1,5-NDA to 1,5-NDI and 50% bitumen was fed to a vacuum distillation in a thin-film evaporator at metering rate 240 g/h. The mixture that contained 38.25% 1,5-NDI monomer was distilled at 160° C. and 0.9 mbar. The monomeric 1,5-NDI was condensed in solid form; the bottoms discharge that was still liquid at this temperature consisted of non-distillable components and bitumen. The bottoms discharge still contained 4.25% 1,5-NDI monomer, which corresponded to a yield of 88.9%.

### Example 2 (Inventive)

A mixture containing 50% residue from the phosgenation of 1,5-NDA to 1,5-NDI and 50% bitumen was fed to a vacuum distillation in a thin-film evaporator at metering rate 257 g/h. The mixture that contained 38.25% 1,5-NDI monomer was distilled at 160° C. and 0.9 mbar. The monomeric 1,5-NDI was condensed in solid form; the bottoms discharge that was still liquid at this temperature consisted of non-distillable components and bitumen. The bottoms discharge still contained 3.19% 1,5-NDI monomer, which corresponded to a yield of 91.7%.

### Example 3 (Inventive)

A mixture containing 60% residue from the phosgenation of 1,5-NDA to 1,5-NDI and 40% bitumen was fed to a vacuum distillation in a thin-film evaporator at metering rate 260 g/h. The mixture that contained 45.90% 1,5-NDI monomer was distilled at 160° C. and 0.9 mbar. The monomeric 1,5-NDI was condensed in solid form; the bottoms discharge that was still liquid at this temperature consisted of non-distillable components and bitumen. The bottoms discharge still contained 9.79% 1,5-NDI monomer, which corresponded to a yield of 78.7%.

### Comparative Example 1

A mixture containing 70% residue from the phosgenation of 1,5-NDA to 1,5-NDI and 30% bitumen was fed to a vacuum distillation in a thin-film evaporator at metering rate 285 g/h. The mixture that contained 48.5% 1,5-NDI monomer was distilled at 170° C. and 1 mbar. The monomeric 1,5-NDI was condensed in solid form; the bottoms discharge that was still liquid at this temperature consisted of 1,5-NDI monomer, non-distillable components and bitumen. The bottoms discharge still contained 31.6% 1,5-NDI monomer, which corresponded to a yield of 34.8%.

### Comparative Example 2

A mixture containing 70% residue from the phosgenation of 1,5-NDA to 1,5-NDI and 30% bitumen was fed to a vacuum distillation in a thin-film evaporator at metering rate 330 g/h. The mixture that contained 53.55% 1,5-NDI monomer was distilled at 160° C. and 1 mbar. The monomeric 1,5-NDI was condensed in solid form; the bottoms discharge that was still liquid at this temperature consisted of non-distillable components and bitumen. The bottoms discharge still contained 18.38% 1,5-NDI monomer, which corresponded to a yield of 65.7%.

### Comparative Example 3

Residue from the phosgenation of 1,5-NDA to 1,5-NDI still containing 62.5% 1,5-NDI monomer was fed without further dilution at a metering rate of 150 g/h at 150° C. and 1 mbar to a vacuum distillation in a thin-film evaporator. The monomeric 1,5-NDI was condensed in solid form; the bottoms discharge that was still liquid at this temperature consisted of 1,5-NDI monomer and non-distillable components. The bottoms discharge still contained 50.5% 1,5-NDI monomer, which corresponded to a yield of 19%.

### Comparative Example 4

Residue from the phosgenation of 1,5-NDA to 1,5-NDI still containing 70-85% 1,5-NDI monomer was fed at 150° C. to an amount of bitumen preheated to 160° C. (30% by weight of the residue) in a tank. 1,5-NDI monomer was continuously distilled out of the mixture obtained while stirring continuously at 2-4 mbar and 160° C. The yield of the 1,5-NDI monomer recovered in this process was 50-60%.

### Discussion of the Examples

It becomes clear from the comparison of the results of the inventive examples with the comparative examples that a distinctly improved yield can be achieved by the process of the invention. Moreover, the remaining residue to be discharged after the distillation is still free-flowing since further oligomerization reactions are very substantially suppressed by the gentle mode of operation, which constitutes an important advantage for the continuous mode of operation. The room temperature solid diisocyanates obtained from the process of the invention are notable for particularly high purity and can be used without restrictions for production of elastomers.

1. A process for recovering room temperature solid diisocyanates from a distillation residue, comprising the following steps:
(i) providing at least one residue containing room temperature solid diisocyanates and
(ii) treating the residue at a temperature of ≥120° C. to <200° C. to obtain room temperature solid diisocyanates,
wherein the residue provided in step (i) contains ≥32% to ≤60% by weight of bitumen and the treatment in step (ii) is effected in at least one thin-film evaporator and/or falling-film evaporator.

2. The process as claimed in claim 1, characterized in that the temperature in step (ii) is ≥130° C. to <190° C., preferably ≥140° C. to ≤180° C. and more preferably ≥150° C. to ≤165° C.

3. The process as claimed in claim 1, characterized in that the room temperature solid diisocyanate is naphthalene 1,5-diisocyanate, naphthalene 1,8-diisocyanate, phenylene 1,4-diisocyanate, tetralin diisocyanate, o-toluidine diisocyanate, durene diisocyanate, benzidine diisocyanate and/or anthrylene 1,4-diisocyanate.

4. The process as claimed in claim 1, characterized in that the residue in the treatment in step (ii) contains ≥0% by weight to ≤4% by weight, based in each case on the total amount of the residue containing room temperature solid diisocyanates, of monomeric diisocyanates having a boiling temperature above the boiling temperature of the room temperature solid diisocyanate.

5. The process as claimed in claim 1, characterized in that the residue has an average residence time in the treatment in step (ii) of ≥1 to ≤15 minutes, in the at least one thin-film evaporator and/or falling-film evaporator.

6. The process as claimed in claim 1, characterized in that the treatment in step (ii) takes place at a pressure of ≥0.4 mbar to ≤4.0 mbar.

7. The process as claimed in claim 1, characterized in that the treatment in step (ii) takes place at a coolant temperature below the melting point of the room temperature solid diisocyanate.

8. The process as claimed in claim 1, characterized in that the treatment in step (ii) gives ≥60% by weight of the room temperature solid diisocyanates that are still present in the residue in step (i).

9. The process as claimed in claim 1, characterized in that the residue containing room temperature solid diisocyanates which is provided in step (i) comes from the phosgenation of the corresponding diamines.

10. A room temperature solid diisocyanate, obtained or obtainable by a process as claimed in claim 1.

11. The use of a thin-film evaporator for recovering aromatic diisocyanates that are solid at room temperature from a residue containing aromatic diisocyanates that are solid at room temperature.

12. A composition comprising at least one room temperature solid diisocyanate as claimed in claim 10 and at least one NCO-reactive compound, preferably at least one polyester polyol.

13. A process for producing an elastomer, in which at least one composition as claimed in claim 12 is cured, optionally while heating.

14. An elastomer produced or producible by a process as claimed in claim 13.
